(19) United States
(12) Patent Application Publication
(10) Pub. No.: US 2006/0122484 A1
(43) Pub. Date: Jun. 8, 2006

(54) Noncontact cargo detector

(75) Inventors: Hideo Itozaki, Tsukuba-shi (JP); Kyoko Kawagishi, Tsukuba-shi (JP); Tadayuki Kondo, Tsukuba-shi (JP); Tadashi Shimizu, Tsukuba-shi (JP); Kenjiro Hashi, Tsukuba-shi (JP)

Correspondence Address: WENDEROTH, LIND & PONACK, L.L.P., 2033 K STREET N. W., SUITE 800, WASHINGTON, DC, 20006-1021, US

(73) Assignee: National Institute For Materials Science, Ibaraki (JP)

(21) Appl. No.: 10/536,001

(22) PCT Filed: Nov. 21, 2003
(86) PCT No.: PCT/JP03/14911

§ 371 Date: Nov. 28, 2005

(30) Foreign Application Priority Data
Nov. 22, 2002 (JP) 2002-340087

(51) Int. Cl. A61B 5/05

(52) U.S. Cl. 600/409

(57) ABSTRACT

A small chemical substance detecting device capable of detecting, by transmitting a radio wave, the NQR of an atom contained in a chemical substance by means of a high-temperature superconducting SQUID magnetic sensor exhibiting a high sensitivity even at a low frequency without unsealing the chemical substance contained in a package or a container. The detecting device can also identify the chemical substance at the same time.

### TECHNICAL FIELD

The invention of this application relates to a detecting device is inspecting a baggage and, more particularly, to a noncontact baggage inspection device capable of inspecting contents of a baggage or hags containing a chemical substance such as a narcotic or an explosive without opening it up.

### BACKGROUND ART

At the time of entry in and exit from a country, generally speaking, checks are made not only by a metal detector but also for chemical substances such as narcotics. A number of baggage inspection devices have been developed (for example, in Cited Publications) for preventing those drugs from being carried into or out of a country.

However, even at present, the detection of chemical substances such as the drugs mostly depends on the sense of smell of dogs. However, few dogs have such a special ability, and it takes a long time to train such dogs. Thus, it is the present situation that no country can sufficiently cope with the increasing smuggling of drugs.

Publication i: JP-A-2001-091661

Publication 2: JP-A-2002-098771

Publication 3: JP-A-2000-028579

Publication 4: JP-A-07-333351

The method for detecting drugs of such chemical substances is exemplified by a nuclear magnetic resonance method (magnetic characteristics), a neutron method (radioactivation characteristics), a chemical method (bonding state of atoms), a biological method (using an antibody bio-film) and so on. Among these, the nuclear magnetic resonance method is excellent for its processing ability.

This nuclear magnetic resonance method generally used is the so-called “NMR method” (Nuclear Magnetic Resonance Spectrometer), and is utilized at present mainly in medical devices such as MRI (Magnetic Resonance Imaging) devices.

The chemical substance detecting method utilizing the nuclear magnetic resonance, the NMR method, utilizes the phenomenon that the nuclear magnetic moment in a chemical substance resonates with a high frequency wave in a magnetic field, and can detect the kind of the chemical substance directly. For this NMR method utilizing the nuclear magnetic resonance, however, a large-sized device is indispensable for generating an intense magnetic field, and the NMR method has a fatal defect in the large size of the device.

Therefore, the invention of this application has an object to solve these problems of the chemical substance detecting devices of the prior arts.

### DISCLOSURE OF THE INVENTION

In order to solve the aforementioned problems, according to the invention of this application, there is firstly provided a noncontact baggage inspection device characterized by comprising: an electromagnetic wave transmitting device including an electromagnetic wave transmitter and an electromagnetic wave transmitting antenna; and a high-temperature superconducting SQUID for receiving the NQR of nitrogen atoms resonating with the transmitted electromagnetic wave. The invention provides secondly a compact noncontact baggage inspection device characterized by comprising a chemical substance detector including an electromagnetic wave transmitting antenna and a high-temperature superconducting SQUID; an electromagnetic wave transmitter; a high-temperature superconducting SQUID controller; and a data processor; thirdly a noncontact baggage inspection device characterized in that the electromagnetic wave transmitting antenna and the high-temperature superconducting SQUID are disposed in a magnetic shield; and in that an endless belt can pass through the inside of the magnetic shield; fourthly a noncontact baggage inspection device characterized in that the magnetic shield is a metal box having a high magnetic permeability; and fifthly a noncontact baggage inspection device characterized in that the cooling medium of the high-temperature superconducting SQUID is liquid nitrogen.

Moreover, the invention of this application provides sixthly a noncontact baggage inspection device characterized in that the transmitted electromagnetic wave is in the radio frequency band of 0.1 to 10 MHz; seventhly a noncontact baggage inspection device characterized in that the electromagnetic wave transmitting antenna has directivity; and eighthly a noncontact baggage inspection device characterized in that a square wave is transmitted from the electromagnetic wave transmitting antenna, and the frequency spectrum obtained by the quick Fourier analysis of the signal detected by the high-temperature superconducting SQUID is compared with the spectral distributions of chemical substances in a database.

### BRIEF DESCRIPTION OF THE DRAWINGS

FIG. 1 is a diagram of an entire noncontact baggage inspection device.

FIG. 2 shows the concept of chemical substance detection utilizing NQR.

FIG. 3 is a diagram showing the relation between frequency and sensitivity.

Here, reference numerals in the drawings designate the following components.
1 Radio Wave Transmitting Antenna
2 Amplifier
3 Radio Wave Transmitter
4 SQUID Electronic Circuit
5 Lock-In Amplifier
6 Data Processor (Personal Computer)
7 SQUID
8 Liquid Nitrogen Container
9 Double Magnetic Shield (with Cylindrical Upper Cover)
10 Double Magnetic Shield (with Mounting Hole In Upper Portion)
11 Belt Conveyor
12 Baggage (Article to Be Inspected)
13 Baggage Entrance
14 Baggage Exit

### BEST MODE FOR CARRYING OUT THE INVENTION

The invention of this application utilizes the phenomenon that when there is a electric field gradient around nitrogen 14 atoms, they resonate along with a low-frequency radio wave, thus allowing detection of the nitrogen 14 atoms existing in a chemical substance. According to the principle of this chemical substance detecting device, a radio wave is transmitted, and the nuclear quadrupole resonance (“NQR”) to a particular frequency of radio wave, intrinsic to nitrogen 14 atoms (14N), is detected.

The invention of this application is characterized by utilizing a superhigh-sensitivity magnetic sensor, the superconducting quantum interference device (abbreviated “SQUID”), for detecting the low-frequency band, which has been especially difficult for the electromagnetic wave detecting coil employed in the prior arts to detect.

The chemical substance detecting device combining the NQR and the high-temperature superconducting SQUID will here be schematically described. First, a radio wave is transmitted from a radio wave transmitter through a radio wave transmitting antenna. For example, when the baggage/article to be inspected is irradiated with the radio wave, the nitrogen 14 atoms existing in the TNT (Trinitrotoluene) employed as an explosive are caused to transmit the NQR signal by the radio wave, and the NQR signal is received by the high-temperature superconducting SQUID being cooled with liquid nitrogen. Then, the data processor compares the NQR signal with the existing resonance frequency thereby to detect the chemical substance contained. The method employing a NQR signal in the invention of this application for detecting a chemical substance uses a principle like that of the NMR (Nuclear Magnetic Resonance Spectrometer) in common use. The essential difference between the NQR and the NMR is that the NMR rises in magnetic field, whereas the NQR rises in an electric field gradient around an atomic nucleus so that NQR is excellent for identifying a substance even in a zero magnetic field.

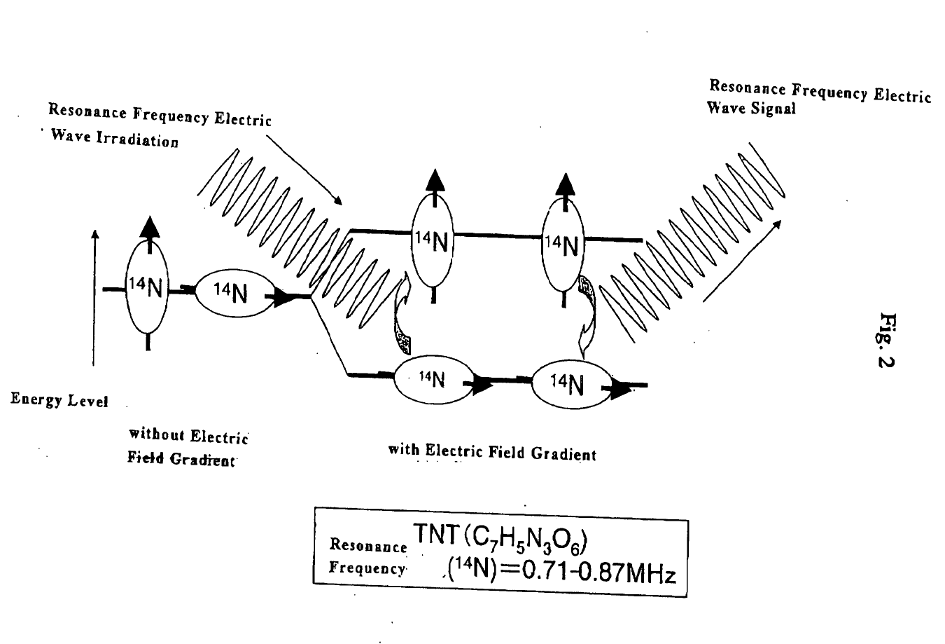

The principle of the NQR to be used in this device is shown in FIG. 2.

As shown in this schematic diagram of FIG. 2, the chemical substance is identified by the resonance vibration intrinsic to a molecule, determined by the unique electric field gradient of the molecule, in this case nitrogen 14. Nowadays, the resonance frequencies intrinsic to several hundreds of thousands of chemical substances have already been examined to make it easy to detect the target chemical substance. The range of the electromagnetic wave usually to be employed for detecting the NQR is a radio wave of 10 MHz or less.

In case a target chemical substance exists nearby, it is detected by bringing the electromagnetic wave transmitting antenna close to the chemical substance. In case the chemical substance is remote, the detection can be made by using a electromagnetic wave transmitting antenna with directivity.

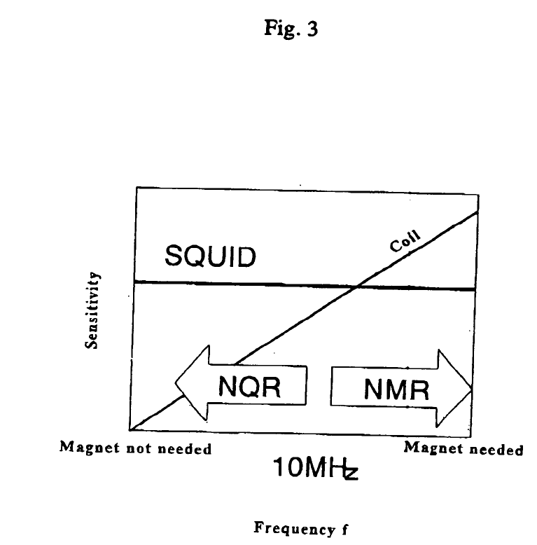

Thus, the chemical substance is detected. However, this resonance frequency of the NQR signal is generally only a few MHz (Megahertz), which is lower than that of the ordinary NMR. This raises the problem that the electromagnetic wave detecting coil usually employed cannot detect the target substance sufficiently. The relation between that frequency (f) and the reception sensitivity are shown in FIG. 5. It is clear from FIG. 3 that the NQR reception sensitivity of the electromagnetic wave detecting coil is seriously lowered in the low-frequency band but that the SQUID sensitivity is constant, independent of the frequency (f).

The invention of this application is proposed to eliminate the above defect, receiving the NQR reliably by means of a high-temperature superconducting SQUID detector in the frequency band of the NQR that the electromagnetic wave detecting coil cannot detect sufficiently.

This SQUID is a high-sensitivity magnetic sensor applying the superconducting quantization, and has a sensitivity one hundred times or more higher than that of the magnetic sensor of the prior art so that it can detect a weak magnetic field one fifty millionth that of the earth's magnetic field.

In the invention of this application, it is preferable to employ not the usual SQUID using helium as a cooling medium but a high-temperature superconducting SQUID. This is because the SQUID of the prior art employing liquid helium as the cooling medium not only is hard to handle but also has the difficulties of high cost for the liquid helium and the large size of heat insulation needed. Thus, it is likely difficult to utilize this SQUID in a portable chemical substance detecting device.

On the contrary, the high-temperature superconducting SQUID is easy to handle and can employ the liquid nitrogen (at 77.3 K: −196° C.) at a low cost so that it can be made small and light, thereby making the noncontact baggage inspection device compactly.

In the invention of this application, therefore, the SQUID is the high-temperature superconducting SQUID which can be cooled down with the liquid nitrogen. However, the superhigh-sensitivity magnetic sensor utilizing this SQUID is so extremely sensitive as to invite a problem that the actually used chemical substance detecting device may pick up environmental noise.

This environmental noise can be efficiently eliminated by providing a magnetic shield for shielding the environmental noise. This magnetic shield is composed of double magnetic shielding plates and configured to expel the NQR signals transmitted from objects other than the target baggage (the inspection target). Thus, the invention of this application detects a chemical substance in a package or a container in a noncontact manner. Here, the chemical substance detecting device of the invention of this application is characterized in that it can identify and detect a plurality of substances simultaneously by changing the frequency. The band of the frequency at this time should not be especially limited but preferably is 0.1 to 10 MHz.

As has been detailed hereinbefore, the chemical substance detecting device of the invention of this application has many features distinguishing it from other chemical substance detecting devices. The excellent features of the invention of this application may be enumerated as follows.

(a) The device can detect the chemical substance itself directly.

(b) The device can detect a plurality of different chemical substances simultaneously by changing the frequency.

(c) The device can be made small and portable.

(d) The device needs no magnetic field for detection.

(e) The device can perform a high-sensitivity measurement by using the SQUID as the sensor.

(f) The device can operate with just a small quantity of liquid nitrogen, by utilizing the high-temperature superconducting SQUID.

The invention of this application has the features described above, and is described below in detail in connection with its Embodiment.

### EMBODIMENT

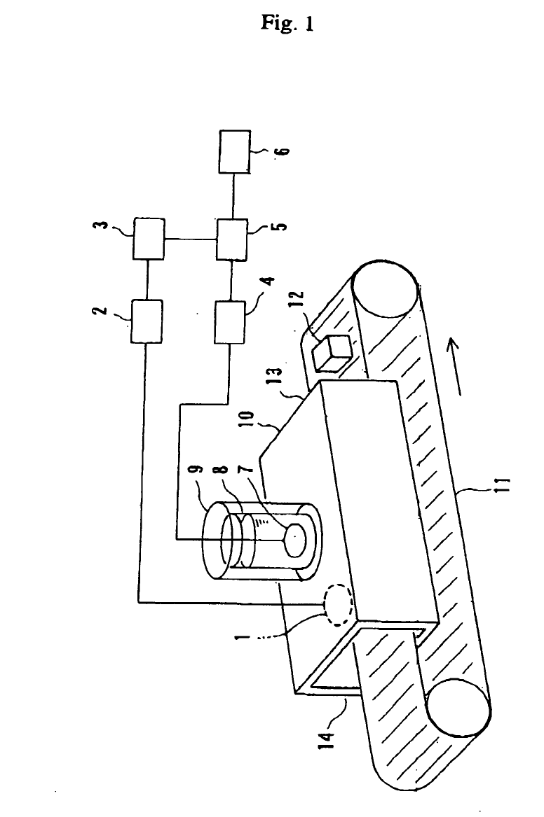

As shown in FIG. 1, this magnetic shield is constituted to include: a rectangular magnetic shield (10) of a double structure provided with an entrance (13) and an exit (14) for a baggage (inspection target) upstream and downstream respectively of a belt conveyor (11 ); and a double-cylinder magnetic shield (9) over an upper through hole in the upper wall of the rectangular magnetic shield (10). The belt of the nonmagnetic belt conveyor (11) can run within the rectangular magnetic shield (10). It is natural that the drive rollers or the motor of the belt conveyor (11) are disposed outside of the magnetic shield (10).

In the cylindrical magnetic shield (9), there is disposed a liquid nitrogen container (8), in which a SQUID (7) is dipped.

A baggage (inspection target) (12) carried on the belt conveyor (11) is introduced from the baggage entrance (13) into the magnetic shield (10). Then, a radio wave transmitted from a radio wave transmitter (3) is amplified by an amplifier (2), and the baggage (or the inspection target) is moved toward the baggage exit (14) while irradiating the baggage (inspection target) with the radio wave transmitted by a radio wave transmitting antenna (1) disposed in the magnetic shield (10).

The NQR signal from the baggage (inspection target) is detected by the SQUID (7) and is outputted from a SQUID electronic circuit (4) to a lock-in amplifier (5) so that the signal of the same frequency as that of the reference signal (taken from Table 1) from the radio wave transmitter (3) is exclusively caught by the lock-in amplifier (5) and outputted to a processor (6). The signal is stored, after about 1,000 integrations, as data in the processor (6). The signal of the transmitter is swept over the band 0.1 to 10 MHz so that the data of the processor (6) are displayed as the spectrum of 0.1 to 10 MHz. These data are collated with the known spectra of explosives or illegal drugs so that the substance is identified. If a substance is identified, a warning is issued.

TABLE 1
NQR Spectrum of Typical Explosives

Unit (MHz)

| TNT | RDX | HMX | Nitrotoluene | |
|---|---|---|---|---|
| Trinitrotoluene | Hexogen | Octogen | p-nitrotoluene | m-nitrotoluene |
| C₇H₅N₃O₆ | C₃H₆N₆O₆ | C₄H₈N₈O₈ | p-C₇H₇NO₂ | m-C₇H₇NO₂ |
| 0.871 | 5.24 | 5.306 | 1.198 | 1.19 |
| 0.8604 | 5.192 | 5.068 | 0.911 | 0.91 |
| 0.845 | 5.047 | 3.737 | | |
| 0.9438 | 3.458 | 3.625 | | |
| 0.838 | 3.41 | 1.564 | | |
| 0.769 | 3.359 | 1.441 | | |
| 0.752 | 1.782 | | | |
| 0.743 | 1.688 | | | |
| 0.716 | | | | |

(Source)
LANDOLT-BÖRNSTEIN
Vol. 20
Neclear Quadrupole Resonance Spectroscopy Data
Editors: K-H Hellwege and A M Hellwege
Springer-Verlag Berlin Heidelberg 1998

In a test of this device, 100 g of the TNT explosive was passed 5 cm below the SQUID. A signal of 1 pt (picotesla) could be caught, and thus the explosive was detected. Likewise, this device can identify various chemical substances such as explosives, poisons, chemicals, and narcotics (such as heroin) so that it can be conveniently utilized for luggage inspections and customs inspections at airports.

### INDUSTRIAL APPLICABILITY

The chemical substance detectors of the prior art were mostly the metal detectors. However, the recent chemical substances to be detected are increasingly non-metallic ones such as plastic bombs. The chemical substance detecting device of the invention of this application can he applied to such chemical substances as plastics and can also be reduced in size. Thus, this chemical substance detecting device can be expected to be used widely as a chemical substance detector in the future.

1. A noncontact baggage inspection device capable of being reduced in size, characterized by comprising: an electromagnetic wave transmitting device including an electromagnetic wave transmitter and an electromagnetic wave transmitting antenna having directivity; a high-temperature superconducting SQUID for receiving the NQR of nitrogen atoms resonating with the transmitted electromagnetic wave; a high-temperature superconducting SQUID controller; and a data processor.

2. (canceled)

3. A noncontact baggage inspection device of claim 1, characterized in that the electromagnetic wave transmitting antenna and the high-temperature superconducting SQUID are disposed in a magnetic shield made of double magnetic shielding plates.

4. A noncontact baggage inspection device of claim 3, characterized in that the magnetic shield is a metal box having a high magnetic permeability.

5. A noncontact baggage inspection device of claim 1, characterized in that a cooling medium of the high-temperature superconducting SQUID is liquid nitrogen.

6. A noncontact baggage inspection device of claim 1, characterized in that the frequency of the transmitted electromagnetic wave is in the radio wave band of 0.1 to 10 MHz.

7. (canceled)

8. A noncontact baggage inspection device of claim 1, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

9. A noncontact baggage inspection device of claim 3, characterized in that a cooling medium of the high-temperature superconducting SQUID is liquid nitrogen.

10. A noncontact baggage inspection device of claim 4, characterized in that a cooling medium of the high-temperature superconducting SQUID is liquid nitrogen.

11. A noncontact baggage inspection device of claim 3, characterized in that the frequency of the transmitted electromagnetic wave is in the radio wave band of 0.1 to 10 MHz.

12. A noncontact baggage inspection device of claim 4, characterized in that the frequency of the transmitted electromagnetic wave is in the radio wave band of 0.1 to 10 MHz.

13. A noncontact baggage inspection device of claim 5, characterized in that the frequency of the transmitted electromagnetic wave is in the radio wave band of 0.1 to 10 MHz.

14. A noncontact baggage inspection device of claim 3, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

15. A noncontact baggage inspection device of claim 4, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

16. A noncontact baggage inspection device of claim 5, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

17. A noncontact baggage inspection device of claim 6, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

18. A noncontact baggage inspection device of claim 9, characterized in that the frequency of the transmitted electromagnetic wave is in the radio wave band of 0.1 to 10 MHz.

19. A noncontact baggage inspection device of claim 10, characterized in that the frequency of the transmitted electromagnetic wave is in the radio wave band of 0.1 to 10 MHz.

20. A noncontact baggage inspection device of claim 9, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

21. A noncontact baggage inspection device of claim 10, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.

22. A noncontact baggage inspection device of claim 11, characterized in that a square wave is transmitted from the electromagnetic wave transmitting antenna, and the frequency spectrum obtained by the quick Fourier analysis of the detected signal of the high-temperature superconducting SQUID obtained is compared with the spectral distribution of a chemical substance obtained from a database.
